(19) United States
(12) Patent Application Publication
(10) Pub. No.: US 2022/0024971 A1
(43) Pub. Date: Jan. 27, 2022

(54) METHOD FOR SYNTHESIZING PEPTIDES

(71) Applicant: STRAINCHEM, Saint-Beauzire (FR)

(72) Inventors: Jean-Jacques YOUTE TENDOUNG, Pont du Château (FR); Audrey SERRE, Clermont-Ferrand (FR)

(73) Assignee: STRAINCHEM, Saint-Beauzire (FR)

(21) Appl. No.: 17/413,807

(22) PCT Filed: Dec. 23, 2019
(86) PCT No.: PCT/FR2019/000218

§ 371 Date: Jun. 14, 2021

(30) Foreign Application Priority Data
Dec. 24, 2018 (FR) 1874091

(51) Int. Cl. C07K 1/04; C07K 5/087

(52) U.S. Cl. CPC C07K 1/042; C07K 5/0812

(57) ABSTRACT

The invention relates to a method for synthesizing peptides or proteins by successively elongating the second end of a peptide chain, the carboxylic acid function (C-terminal) or the amine function Na of which is attached to an anchoring molecule that is soluble in an apolar solvent, characterized in that said anchoring molecule comprises a polyolefin chain with at least 10 monomer units, and preferably between 15 and 50 units.

### TECHNICAL FIELD OF THE INVENTION

The invention relates to the field of organic chemistry and more specifically to the synthesis of peptides or proteins from amino acids. The invention concerns a method for synthesizing peptides in vitro, in the liquid phase, which does not use a solid substrate or an insoluble resin. This method is based on using a new family of anchoring molecules, that is, derivatives of polyolefins or oligomers of polyolefins or polyalkenes, which, bonded to an amino acid or an amino acid derivative, exhibit good solubility in apolar solvents, but exhibit poor solubility in water. This method makes it possible to obtain peptides that are purer and/or easier to purify than the known methods using a solid substrate or a liquid substrate. It is easy to automate.

### PRIOR ART

The pharmacology of peptides is of tremendous medical and economic interest, but from a scientific standpoint, it remains in the shadow of protein and genetic biochemistry. Despite the immense success of insulin, a natural protein made of 51 amino acids, the number of peptides that have been developed as active pharmaceutical ingredients has remained modest through the end of the 20th century. However, some thirty new active ingredients consisting of peptides received marketing authorizations between 2000 and 2016, and a significant number of peptides are undergoing clinical and preclinical trials (see the publication by A. Henninot et al., “The Current State of Peptide Drug Discovery: Back to the Future?” published in J. Med. Chem., 2018, 61, 4, 1382-1414). Most of these medication candidates are natural peptides known for their specific function, others are derived from natural sequences, and others still are totally synthetic.

In the case of insulin, which is administered to millions of patients, synthesis by genetically modified organisms is the preferred pathway for obtaining sufficiently pure industrial quantities. For many other therapeutic peptides, ab initio synthesis (that is, synthesis from individual amino acids) is the main method used to obtain necessary quantities. Given this context, peptide purity is a major technical and economic challenge for the chosen method of synthesis. At the same time, the environmental factor is becoming an increasingly important societal concern. For this reason, chemists are looking for a method that is consistent with this criterion (see the publication by A. Isidro-Llobet et al., “Sustainability Challenges in Peptide synthesis and Purification: From R&D to Production,” published in J. Org. CHEM., 2019, 84, 4615-4628). The purification of a peptide (which is generally done by high-performance liquid chromatography, HPLC) can account for a large proportion of the cost price of a finished product, but can also generate waste. There are two major methodologies of peptide synthesis which are distinguished by the physical state of the phase in which the chemical reactions take place: the liquid phase (this synthesis is also referred to as “synthesis in solution”) and the solid phase (this synthesis is also referred to as “solid-phase synthesis”).

All amino acids have at least two reactive chemical functions: the amine function (Nα center) and the carboxylic acid function (C-terminus). Certain amino acids also have lateral chains capable of reacting with Nα centers or C-termini. The key to a peptide synthesis strategy is in the choice of protecting groups by which the reaction centers are protected during certain steps of the method. Consequently, each addition of an amino acid requires a cycle of steps: protection—activation/coupling—deprotection. At the end of synthesis, the protecting groups are cleaved to generate the target peptide. Consequently, depending on the physical state of the phase in which the peptide synthesis takes place, the common basic cycle: protection—activation/coupling—deprotection is invariable, and only the protecting group used differs. More specifically, such a cycle comprises several steps:
Protection of the amine function (Nα) of the amino acid by a protecting group that can be cleaved after the amino acid condensation reaction;
If necessary, protection of the lateral chain with a protecting group that can be cleaved at the end of peptide synthesis;
Activation of the carboxylic acid function of the protected amino acid Nα, then the condensation thereof with an amino acid derivative or a peptide of which the amine function Nα is free and against which the carboxylic acid function is protected;
At the end of the iteration (activation/coupling—deprotection) of all the amino acids in the sequence, the target peptide is obtained by total deprotection of the protecting groups.

The protection—activation/coupling—deprotection cycle is explained here for the example of the synthesis of a tripeptide.

According to the solution strategy called “Boc” shown in reaction diagram 1, a first, a second, and a third amino acid (referred to here as AA1, AA2, and AA3) are provided. The amine function (Nα) of amino acid AA2 is temporarily protected by a tert-butoxycarbonyl group (commonly called “Boc”). We then activate the carboxylic acid function (C-terminus) of said protected amino acid, which is condensed with the free (i.e. not protected) amine function (Nα) of a methyl ester of amino acid AA1, which generates a dipeptide. Next, the amine function (Nα) of amino acid AA2 of the resulting dipeptide is deprotected (i.e. protecting group Boc is cleaved with an acid, such as trifluoroacetic acid). The amine function (Nα) is protected against amino acid AA3. We then activate the carboxylic acid function (C-terminus) of amino acid AA3, which is bonded to the amine function (Nα) of amino acid AA2 (of the deprotected dipeptide) to generate the protected tripeptide. After deprotection of the Boc group carried by AA3, a tripeptide is obtained. This process comprises a minimum of five reaction steps, including two steps (activation/coupling—deprotection) for each additional amino acid added to the peptide.

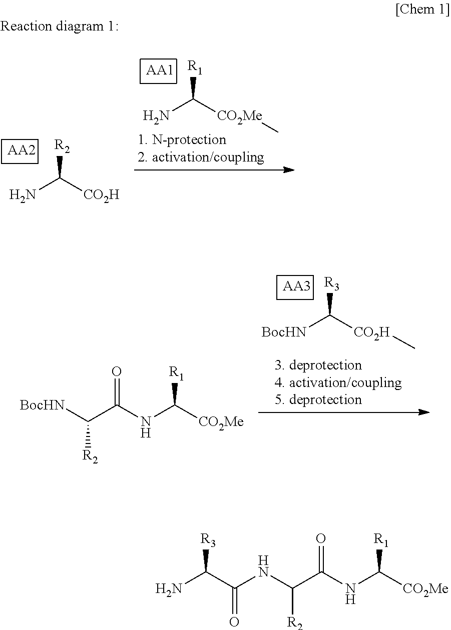

This method can take place with anchoring of amino acid AA1 to a solid substrate or to an organic solubilizing molecule in the liquid phase.

The fluorenylmethoxycarbonyl can be used as a protecting group for the amine function according to the so-called “Fmoc” strategy shown in reaction diagram 2. This strategy is particularly well suited for synthesis on a solid substrate. A first, a second, and a third amino acid (AA1, AA2, and AA3) are provided. It all starts with protection of the amine function (Nα) of amino acid AA1 with a fluorenylmethoxycarbonyl group (commonly referred to as “Fmoc”), then the anchoring thereof to a solid resin (symbolized in reaction diagram 2 by the black circle) via the carboxylic acid function (C-terminus) thereof, forming a covalent bond link covalent of the ester or amide type with a chemical function of the functionalized resin (alcohol or amine). The amine function (Nα) of the amino acid anchored to the resin is deprotected. The amine function (Nα) of amino acid AA2 is then protected with an Fmoc protecting group. The resulting derivative is bonded by activation of the carboxylic acid (C-terminus) thereof to the amine function (Nα) of amino acid AA1 anchored to the resin, which generates an N-protected dipeptide anchored to the resin. The Fmoc group of the dipeptide (on AA2) is cleaved. Protection of the amine function (Nα) of amino acid AA3 by an Fmoc group is followed by activation of its carboxylic acid (C-terminus) then the resulting species is made to couple to the anchored dipeptide having the free amine function, thus generating an N-protected tripeptide, which remains attached to the solid substrate by the acid function (C-terminus) of its amino acid AA1. This method comprises a minimum of seven reaction steps, including two (activation/coupling—deprotection) for each additional amino acid added to the peptide.

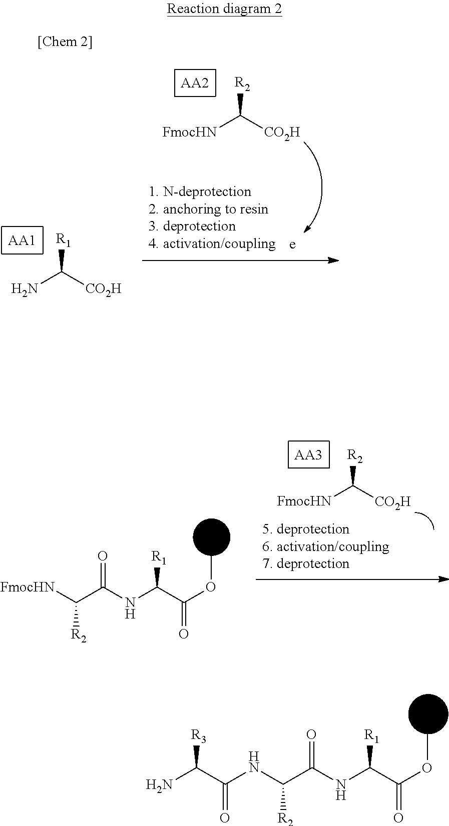

These two synthetic pathways are well known to a person skilled in the art (see Section 7-5 of the manual titled “Biochimie” [Biochemistry] by D. Voet and J. G. Voet, 2^{nd} edition, Brussels 2005). They can be implemented in the liquid phase or on a solid substrate (in this case the amino acid is attached to a solid substrate, i.e. Merrifield synthesis). In practice, the amino acids are supplied in the Fmoc- or Boc-protected state and are used directly in the activation/coupling reactions in this protected state.

During Liquid-Phase Peptide Synthesis (LPPS) all the reactions take place in a homogeneous solution. This methodology has been described by Bodansky and du Vigneaud (J. Am. Chem. Soc., 1959, 51, 5688-5691). The carboxylic acid function (C-terminus) of the starting amino acid is protected in the form of a methyl ester and the following amino acids are condensed successively after their amine function (Nα) has been protected by a benzyloxycarbonyl group and their carboxylic acid function (C-terminus) has been activated by a nitrophenyl ester. The synthetic intermediates are all purified by precipitation or washing with water (extraction). This peptide synthesis methodology is length and tedious and generates peptides at a low yield. As an example, the synthesis of ACTH has an overall yield of about 7%, as described by Schwyzer and Sieber (Helv. Chim. Acta 1966, 49, 134-158).

A modification of this methodology has been reported by Beyermann et al. (Ree. Tray. Chim. Holland 1973, 92, 481-492). It consists in protecting the carboxylic acid function (C-terminus) of an amino acid or a peptide in the form of a benzyl ester and conducting the coupling (or condensation) reaction in the presence of excess N-protected amino acid (Nα) anhydride in an effort to improve the yield. Lastly, although the yields of the coupling reactions are improved, there is a loss of organic phase solubility when the resulting peptide reaches approximately five amino acids.

To make gains in solubility, the amino acid or the protein can be bonded to a so-called solubilizing protecting group, such as the (phenylsulfonyl)ethyl ester (OPSE) described in EP 0 017 536 (CM Industries). Indeed, this protecting group allows the amino acid or the synthesized peptide to be solubilized in N,N-dimethylformamide. After each iteration (activation/coupling—deprotection), purification is done by precipitation and filtration of the solids, which involves some operating difficulties.

An approach making use of a linear non-crosslinked polymer such as polyethylene glycol (PEG) in order to keep the amino acid or peptide in solution, has been described by Mutter and Bayer (Nature 1972, 237, 512-513). In this case as well, the byproducts are eliminated by precipitation or ultracentrifugation.

More recently, EP 2 612 845 A1 and US 2014/0296483 (Ajinmoto Co., Inc.) described new protecting groups (or anchoring molecules) for solubilizing the amino acid and the peptide. In this way, depending on the anchoring molecule used, purification takes place either by simple water washing or by precipitation and filtration. Thanks to these highly lipophilic protecting groups of the carboxylic acid function, bivalirudin, an anticoagulant consisting of 20 amino acid residues, was prepared with an overall yield of 73% and a purity of 84% (see D. Takahashi et al., Angew. Chem. Int. Ed., 2017, 56, 7803-7807). Despite the advantages of this technique, its limit, i.e. the number of amino acid residues likely to be anchored, remains unknown. In addition, the financial and environmental costs of these anchoring molecules are a drawback.

Other amino acid coupling strategies are known. As an example, bi-functional groups that both activate the carboxylic acid function (C-terminus) and protect the amine function (Nα) of the amino acid by forming highly-reactive intermediate cyclic structures, can be used. Generally, they readily react with the amine function (Nα) of a second amino acid derivative to form a dipeptide. Unfortunately, the resulting species are very reactive and often an undesirable polymerization reaction ensues. Of the bi-functional groups that have been described, let us mention phosgene (see R. B. Woodward and C. H.

Schramm, J. Am. Chem. Soc., 1947, 69, 1551-1552), dichlorodimethylsilane (see S. H. Van Leeuwen et al., Tetrahedron Letters 2002, 43, 9203-9207), hexafluoroacetone (see J. Spengler et al., Chem. Rev., 2006, 106, 4728-4746), and formaldehyde (see J. M. Scholtz, P. A. Bartlett, Synthesis 1989, 542-544).

Solid-phase peptide synthesis has been described by Merrifield (J. Am. Chem. Soc., 1963, 85, 2149-2154). This consists in attaching the carboxylic acid function (C-terminus) of the first amino acid or the peptide to an insoluble resin (substrate). Consequently, the reagents are used in excess to ensure the total conversion of the activation/coupling steps. The purifications take place by simple filtration and washing of the resin. Although this technique is simpler and can be automated, there are numerous drawbacks, such as the cost and loss of the reagents used in excess, and the lack of homogeneity of the synthesized peptides as it is practically impossible to obtain homogeneous peptides: the system is said to be degenerate. In addition, purification by high-performance liquid chromatography for preparing these heterogeneous peptides is burdensome because of its high consumption of solvents and its unacceptable ecological cost.

Knowing that each reaction step is rarely quantitative and therefore generates losses that add up throughout the synthesis process, and knowing that each step involves reagents in solution (coupling agents and secondary products) whose elimination is problematic, there is an interest in having a simpler method producing highly pure peptides at a lesser and more ecologically-compatible production cost. The method should be suitable for all types of natural amino acids and for a broad spectrum of non-natural amino acids. Such is the purpose of the present invention.

It should be emphasized that in the case of a therapeutic peptide, a yield of less than 100% not only means the loss of reagents, but also the formation of secondary products which may be difficult to separate from the target peptide; to the extent that one wishes to obtain the purest possible peptides so as to clearly characterize their biological effects, either one accepts the additional cost incurred by purification, or one accepts the presence of impurities likely to cause a risk of error in assessing the observed biological effects.

The problem that the present invention aims to solve is to present a more ecological method for synthesizing purer peptides or proteins at a higher yield and at a lesser cost, which can be automated. The method must be suitable at least for all natural amino acids and preferably for a broad spectrum of non-natural amino acids. The method should not involve anchoring molecules or substrates that are costly or difficult to synthesize.

### SUBJECT MATTER OF THE INVENTION

The present invention proposes solving the difficulties remaining in the prior art by using polyolefins or oligomers of polyolefins or polyalkenes to produce highly-pure peptides or proteins in the liquid phase. Indeed, the inventors have found that using polyolefins, and particularly polyisobutene (PIB) derivatives, as anchoring molecules or a liquid substrate, allows amino acids to be solubilized and peptides to be synthesized in an organic solution (halogenated and non-halogenated solvents), while facilitating the purification thereof by simple extraction or washing, in the present case with water or a water/ethanol or water/acetonitrile mixture, or by simple filtration. In addition, some of these anchoring molecules (particularly certain polyisobutene (PIB) derivatives) are commercially available products or they are easy to synthesize directly and at low cost.

The subject matter of the invention is thus a method for synthesizing peptides or proteins by successively elongating the second end (Nα) of a peptide chain of which the first end is attached to an anchoring molecule that is soluble in an apolar solvent by means of the carboxylic acid function (C-terminus) thereof, characterized in that said anchoring molecule includes a polyolefin chain with at least 10 monomer units and preferably between 15 and 50 units. It is advantageously functionalized at one of the ends thereof to allow the first amino acid to be anchored.

Said anchoring molecule is advantageously a polyolefin.

Said anchoring molecule advantageously includes only one polyolefin chain; the polyolefin chain can thus be obtained by a simple polymerization reaction (of the isobutene).

Said anchoring molecule may comprise in each of its units identical or non-identical alkyl groups that are preferably selected from the group consisting of methyl and ethyl. Said polyolefin chain advantageously has an average molecular weight by mass of between 600 and 20,000, and preferably between 700 and 15,000. Said polyolefin chain may comprise a number of unsaturated carbon-carbon bonds not exceeding 5% and preferably not exceeding 3%. It is preferably a polyisobutene chain.

In an advantageous embodiment of the method according to the invention, said anchoring molecule includes a polyolefin chain (or is a polyolefin chain) that is terminated by a group selected from the following group consisting of:
a —X function, where X is selected from the group consisting of: —OH, —NH₂, —SH;
a —Z—C₆H₄X¹ function, where
   Z is O or is absent,
   X¹ is selected from the group consisting of: —OH, —NH₂, —SH, —NH—NH₂, —CXRR¹, —C₆H₃R′(CRX)
   where X is selected from the group consisting of —OH, —NH₂, —SH, and R is selected from the group consisting of —H, Aryl, Heteroaryl, and R′ is selected from the group consisting of —H, -Alkyl, —O-Alkyl, -Aryl, —O-Aryl, Heteroaryl, —O-Heteroaryl,
a —CR″═CH—CHX function or a —CR″H—CH═CH—CHX function where X is selected from the group consisting of —OH, —NH₂, —SH, and R″ is methyl or ethyl.

This group then represents the functionalization of the polyolefin chain.

Said first end of said peptide chain is a first amino acid AA1 unit; the anchoring molecule bonds to this first amino acid AA1, either to its carboxylic acid function (C-terminus) or to its amine function (Nα). Said peptide chain is formed of n amino acid units; the second end thereof is another amino acid unit AAn. As the method proceeds, the peptide chain lengthens by successive elongation and, during each elongation step, another amino acid unit AA(n+1) is added to said second end.

The amine function (Nα) of the amino acids involved in the method according to the invention can be protected by a Boc or Fmoc group, or by any other appropriate protecting groups.

Natural and/or non-natural and/or synthetic amino acids can be used in said peptide chain.

The method according to the invention comprises at least one step wherein said peptide chain is attached to said anchoring molecule and is separated from the reaction medium by extraction in an apolar solvent.

The method according to the invention makes it possible to obtain highly pure peptides or proteins, which are cleaved from their anchoring molecule after the last peptide chain elongation step so as to be used as intended, for example as an active ingredient for preclinical or clinical trials.

### DETAILED DESCRIPTION

### Definitions

In the present invention, an “amino acid” is understood as encompassing: natural amino acids and non-natural amino acids. “Natural” amino acids comprise the L-form of amino acids that can be found in natural-origin proteins, that is: alanine (Ala), arginine (Arg), asparagine (Asn), aspartic acid (Asp), cysteine (Cys), glutamine (Gin), glutamic acid (Glu), glycine (Gly), histidine (His), isoleucine (ILe), leucine (Leu), lysine (Lys), methionine (Met), phenylalanine (Phe), proline (Pro), serine (Ser), threonine (Thr), tryptophan (Trp), tyrosine (Tyr), and valine (Val).

“Non-natural” amino acids comprise the D-form of the natural amino acids defined above, the homo forms of certain natural amino acids (such as: arginine, lysine, phenylalanine, and serine), and the nor-forms of leucine and valine. They also include non-natural amino acids, such as:

Abu=2-aminobutyric acid CH₃—CH₂—CH(COOH)(NH₂)
iPr=Isopropyl-lysine (CH₃)₂C—NH—(CH₂)₄—CH(COOH)(NH₂)
Aib=2-aminoisobutyric acid
F-trp=N-formyl-tryptophan
Orn=ornithine
Nal2=2-naphthylalanine

The “non-natural” amino acids also comprise all synthetic amino acids. The term “protected amino acid” is also used here to refer to temporarily protected amino acids, particularly as described above; for example, the amine function (Nα) can be protected by an Fmoc, Boc, or benzyl group, or any other appropriate protecting groups.

The peptide preparation method of the invention uses polyolefins, or more specifically oligomers of polyolefins (with polyolefins also being called polyalkenes), and their derivatives as anchoring molecules or protecting groups, either for the carboxylic acid function (C-terminus) of the amino acid or the peptide, or for the amine function (Nα) of the amino acid or the peptide, or for the lateral chain of said amino acid or peptide (in the form of ester, amide, ether, or thioether bonds, or any other functions) in the liquid phase. The polyolefin molecules comprise a chain of carbon atoms connected by single bonds. They may comprise branches consisting of identical or different alkyl groups, but preferably identical. Preferably, polymers with at least 10, and preferably 15 to 50, monomer units are used. Homopolymers are preferred, but copolymers can also be used, in which case the number of unsaturated bonds in the chain of carbon atoms advantageously does not exceed 5% and preferably does not exceed 3%.

They are preferably polyisobutenes (PIBs), a class of polymers known since the thirties of last century, but polypropylene derivatives can also be used. These anchoring molecules are preferably used in the method according to the invention in the form of functionalized derivatives, as will be explained in greater detail below.

According to the invention, these anchoring molecules are attached to the carboxylic acid function of an amino acid (C-terminus) or to the amine function (Nα) by a covalent bond of the amide, ester, benzyl, or allyl type, or any other function. This assumes that the anchoring molecules are involved in a suitably functionalized form which, in this description, are called “PIB derivatives,” bearing in mind that here this term also encompasses the derivatives of anchoring molecules that are not derivatives of polyisobutene, but which are derivatives of other polyolefins according to the definition given above. As a general rule, this functionalization of the anchoring molecule is a terminal functionalization, preferably at one of the ends of the chain of carbon atoms; it will be described below.

The oligomers of polyolefins used as anchoring molecules are typically characterized by an average molecular weight by mass, but “pure” oligomers including identical molecules of a given chain length may also be used. This reaction between the PIB derivative and the amino acid leads to a product characterized in that when the PIB derivative is attached to an amino acid or an amino acid derivative, possibly having a protected lateral chain, a molecule with a low solubility in water (<30 mg/ml) is obtained.

More specifically, the method for synthesizing peptides, possibly protected peptides, in the liquid phase (solution) according to the invention is characterized by the fact that an amino acid or a peptide is solubilized in an organic medium by a PIB derivative attached to the carboxylic acid function (C-terminus) or to the amine function (Nα) of the amino acid or the peptide.

The PIB derivative acts as an anchoring molecule or liquid substrate of the amino acid or the peptide, which is synthesized by the successive attachment of amino acids to said amino acid attached to this anchored molecule. Consequently, the anchoring molecule also serves as a protecting group during the synthesis of the peptide over successive iterations.

The potentially protected amino acid or peptide, anchored to a PIB molecule, is characterized in that the carboxylic acid function (C-terminus) or the amine function (Nα) of said amino acid or peptide is attached by a covalent bond of the ester, amide, benzyl, or allyl type, or any other chemical functions with a lipophilic PIB derivative, yielding a very low solubility in water (<30 mg/ml). It is in this way that, in the method of the invention, said anchoring molecule, which is preferably a PIB derivative, acts as a liquid substrate, for the synthesis of peptides or proteins.

This derivation of the amino acid (Nα, protected or not protected) or of the peptide (Nα, protected or not protected) with the PIB derivative significantly increases the solubility of said amino acid or of said peptide in an apolar organic liquid phase. More specifically, these amino acids and these peptides become soluble in organic solvents, such as halogenated solvents (methylene chloride, chloroform), ethyl acetate, tetrahydrofuran, cyclohexane, hexane(s), or aromatic solvents such as benzene or toluene. Consequently, the amino acids and peptides attached to a PIB derivative have a high distribution ratio for the organic phase during an extraction/decantation in the presence of water or a water/ethanol or a water/acetonitrile mixture, thus allowing for the simple and quick purification thereof.

The present invention also proposes a method for synthesizing (protected or non-protected) peptides in the liquid phase, characterized in that we start with an amino acid or a peptide in solution (or an amino acid or peptide derivative in solution), which will be attached to one of the anchoring molecules as defined earlier, by means of the carboxylic acid function (C-terminus) or the amine function (Nα) of the initial amino acid or peptide derivative, and we add or condense the next amino acids or peptides, which are protected on their amine function (Nα) and possibly on their lateral chain, after activation of their carboxylic acid function (C-terminus).

Activation of the carboxylic acid function (C-terminus) of the Nα-protected amino acid or of the peptide may be done by any known synthesis technique via the formation of an anhydride or by means of various reagents such as: carbodiimide, acid chloride, alkyl chloroformate, or any other technique for activating an Nα-protected amino acid.

The method for synthesizing peptides according to the invention is also characterized in that the amino acids or peptides to be condensed are then added. These amino acids or peptides are activated on their acid function and are protected on their amine function (Nα), and, if necessary, also protected on their lateral chain.

Here, we present the method of the invention step-by-step using an example in which an H-Tyr-Gly-Phe-OH tripeptide is targeted, using either Boc or Fmoc protecting groups.

Reaction diagram 3 shows the first step in coupling the acid function of the first amino acid (AA1), in the present case phenylalanine (Phe), to a PIB derivative functionalized by a phenol. Amino acid AA1 is used in the protected state by an Fmoc group.

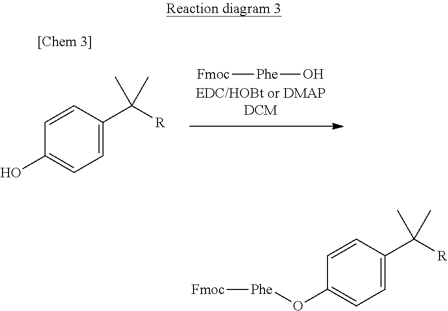

In this reaction diagram, DCM is dichloromethane, EDC is 1-ethyl-3-(3-dimethylaminopropyl)carbodiimide, HOBt is hydroxybenzotriazole, and DMAP is 4-dimethylaminopyridine.

Reaction diagram 4 shows the variant in which AA1 is used in the protected state by a Boc group.

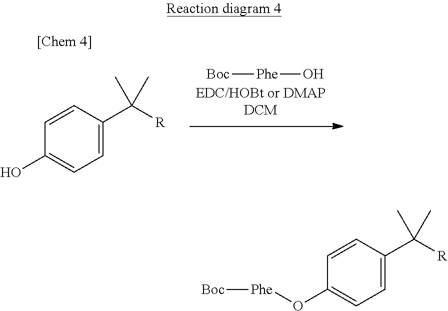

In a second step the protecting group (Fmoc or Boc) is cleaved to release the amine function (Nα) of AA1. This so-called deprotection step is illustrated in reaction diagram 5 in the case of Fmoc, and reaction diagram 6 in the case of Boc. It leads to a molecule which we refer to here as PIB-AA1.

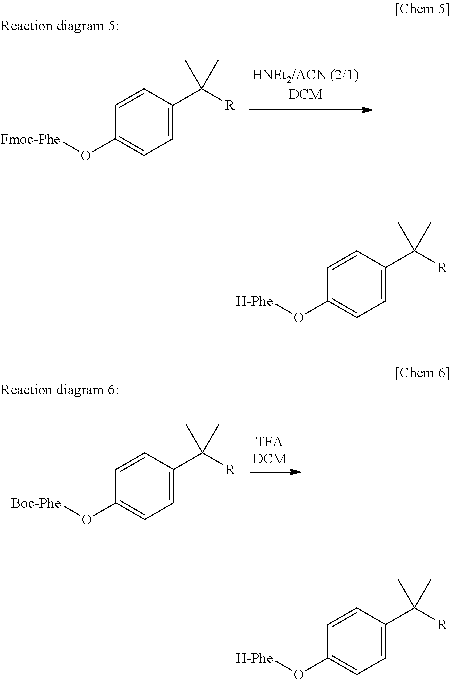

ACN is acetonitrile, HNEt2 is diethylamine, and TFA is trifluoroacetic acid. In a third step, the first step is repeated by adding the second amino acid (referred to here as AA2), in the present case glycine (Gly), to molecule PIB-AA1, in the protected state (Boc or Fmoc); the acid function (C-terminus) of AA2 (the Nα function of which is protected) bonds to the N-terminus function of PIB-AA1. In a fourth step the Nα function of PIB-AA1-AA2 is deprotected, generating the dipeptide PIB-AA1-AA2 anchored to its substrate.

In a fifth step, the first step is repeated by adding the third amino acid (referred to here as AA3), in the present case tyrosine protected on its lateral chain by a terf-butyl group to the molecule PIB-AA1-AA2, in the protected state (Boc or Fmoc); the acid function (C-terminus) of AA3 (the Nα function of which is protected) bonds to the Nα function of PIB-AA1-AA2. In a sixth step, the Nα function of PIB-AA1-AA2-AA3 is deprotected and the anchored tripeptide shown in reaction diagram 7 is obtained.

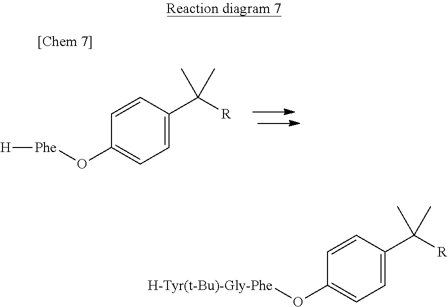

One can easily see that, through successive iterations, this method makes it possible to add successive amino acids to the amino acid and then to the peptide attached to the PIB derivative, in order to thus obtain a peptide having the desired sequence. With the peptide attached to the liquid substrate, it can be separated at any time, particularly after the last iteration, from any polar products by extraction in an apolar solvent. At the end of this elongation sequence, the peptide can be detached from the polymer substrate; the peptide thus loses its solubility in an apolar phase and it can be separated from the polymer substrate in order to be used as intended.

This reaction for detaching the peptide from its liquid substrate, is shown in reaction diagram 8 for the case of the tripeptide in reaction diagram 7. A mixture of trifluoroacetic acid (TFA), of triisopropylsilane (TIPS), and water can be used.

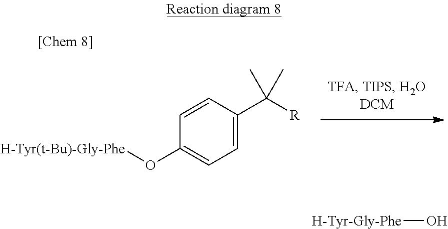

Diagram 9 shows a certain number of PIB derivatives with their functionalization which are suitable as liquid substrates for carrying out this invention.

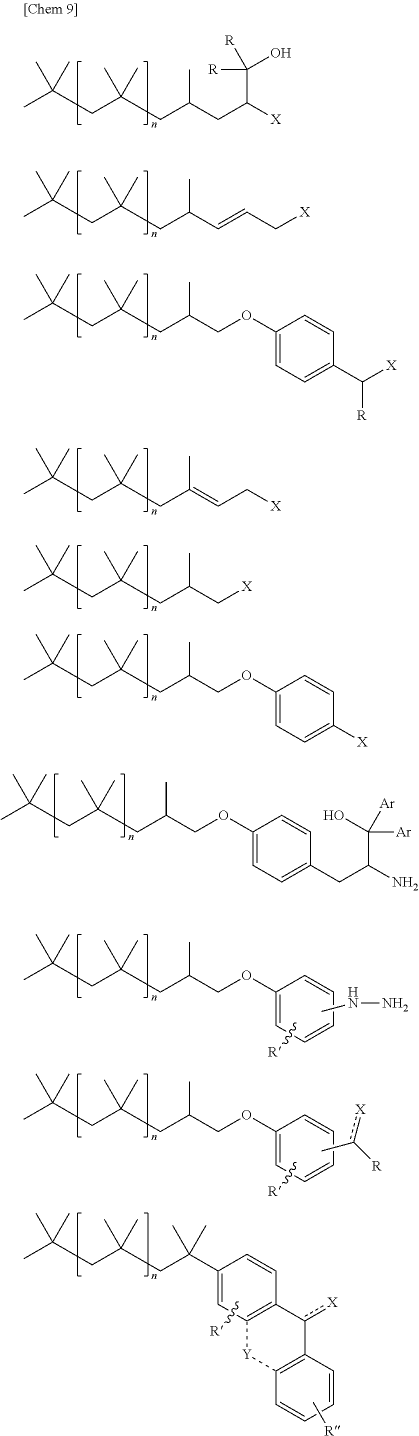

In these formulas:
X is a group selected from the group consisting of: OH, NH₂, NH—NH₂, SH;
R is a group selected from the group consisting of: H, aryl, heteroaryl;
R′ is a group selected from the group consisting of: H, alkyl, O-alkyl, heteroaryl, O-(heteroaryl);
R″ is a group selected from the group consisting of: H, alkyl, O-alkyl, heteroaryl, O-(heteroaryl),
Y is a group selected from the group consisting of: O, CH₂CH₂.
the number n is a whole number which is typically greater than 10 and advantageously between 15 and 50.

In particular, X can be a free amine, a hydrazine, an alcohol, a thiol, or a phenol.

In an advantageous embodiment, the average molecular weight by mass of the anchoring molecules, excluding the terminal functionalization (for example, —X, —Z—C₆H₄X¹, or CR″═CH—CHX as defined above), is between 600 and 20,000, and preferably between 700 and 15,000. Beyond a molecular weight of about 20,000 these molecules are excessively viscous, which could limit their solubility in the solvents (halogenated or non-halogenated solvents) used for the activation/coupling step.

Certain PIB derivatives that can be used as part of this invention are commercially available as ligands for homogeneous catalysis. As an example, it is possible to use 2-Methyl-3-[polyisobutyl (12)]propanol (average molecular weight by mass 757, including the terminal functionalization) or 4-[Polyisobutyl(18)]phenol (average molecular weight by mass 1104, including the terminal functionalization), which are distributed under item numbers 06-1037 and 06-1048, respectively, by Strem Chemicals. These two molecules are polyisobutene derivatives in which the chain is terminated by a —CH₂—C(CH₃)(H)—CH₂—OH group (i.e. isopropyl alcohol) and by a —CH₂—C(CH₃)₂—C₆H₅—OH (i.e. phenol) group, respectively.

The preferred anchoring molecules, i.e. polyisobutene derivatives, can be prepared from biobased isobutene. The concept of biobased content is defined in ISO Standard 16620-1:2015 “Plastics—Biobased content—part 1: General principles,” particularly by a definition of the terms “biobased synthetic polymer,” “biobased synthetic polymer content,” “biobased carbon content,” and “biobased mass content,” as well as in ISO Standards 16620-2:2015 “Plastics—Biobased content—part 2: Determination of the biobased carbon content,” and 16620-3:2015 “Plastics—Biobased content—part 3: Determination of biobased synthetic polymer content,” for the methods for determining and quantifying the biobased characteristics.

Advantageously, the anchoring molecules have a biobased carbon content greater than 90%, preferably greater than 93%, and even more preferably greater than 95%.

The method of the invention has numerous advantages.

A first advantage is that it enables peptide production in the liquid phase in which the peptide (Nα-protected or not) attached to the anchoring molecule, as defined earlier, remains in organic solution.

A second advantage is that it makes it possible to obtain highly pure peptides by simple washing with water or a water/ethanol or water/acetonitrile mixture, or by filtration, thus eliminating the by-products (salts, acids, or any other molecular species occurring, for example, during deprotection of the amine function) that are not attached to the derivative of polyolefins or oligomers of polyolefins or polyalkenes and reagents in excess in the organic phase. Organic solvents such as cyclohexane, heptane(s), and hexane(s) which have flash points of less than 15° C. are appropriate for solubilizing the derivatives of polyolefins or oligomers of polyolefins or polyalkenes during extraction or washing. The method according to the invention therefore avoids all the purification steps required in the methods of the prior art.

A third particularly important advantage is that the method of the invention is able to synthesize peptides and even proteins by adjusting the length of the derivative of polyolefins or oligomers of polyolefins or polyalkenes by making them more lipophilic.

Another advantage is the possibility of controlling the purity of the peptide at any time during synthesis by taking an aliquot followed by an analysis using the various techniques known to a person skilled in the art (such as mass strectrometry, high-performance liquid chromatography, proton nuclear magnetic resonance, or carbon-13).

Yet another advantage is that the preferred anchoring molecules, i.e. polyisobutene derivatives, can be prepared from biobased isobutene as already explained above.

Other advantages are the possibility of automating the method of the invention and the possibility of recycling the anchoring molecules (polyolefins or oligomers of polyolefins or polyalkenes). Indeed, once the series of iterations for obtaining the sequence of the target peptide is completed, the peptide is deprotected from its protecting groups and ultimately the anchoring molecule by one of the reactions customarily used in peptide synthesis (such as hydrolysis, saponification, and hydrogenolysis), which releases the anchoring molecule. The anchoring molecule can thus be recycled. Thanks to their high purity, the peptides or proteins produced by this method may be used as pharmaceutical products (medications and vaccines), cosmetic products, phytosanitary products, or agrifood products, or for access to any one of these products.

### EXAMPLES

These examples illustrate embodiments of the invention, but do not limit the scope thereof. Examples 1 and 2 illustrate two variants of the coupling reaction of the first amino acid (AA1) of the target peptide (used in the reaction in the Nα-protected form by Fmoc or Boc) with a liquid substrate (in the present case a PIB derivative). Examples 3 and 4 illustrate two variants of the deprotection of the next amino acid (AA2) used in the reaction in the Nα-protected form by Fmoc or Boc (referred to here as “Fmoc derivative” ou “Boc derivative”). Example 5 illustrates the detachment of the peptide from the anchoring molecule according to reaction diagram 8 above.

These examples use chlorinated solvents, i.e. DCM. Similar results have been obtained with less harmful solvents, such as: tetrahydrofuran, 2-methyltetrahydrofuran, and ethylene carbonate, pure or mixed.

### Example 1: Coupling Reaction Using EDC/HOBt

The Nα-protected amino acid (Fmoc-AA-OH or Boc-AA-OH) (1.3 mmol) was dissolved in DCM (5 mL) with magnetic stirring and in a nitrogen atmosphere, then cooled to 0° C. in an ice bath. The 4-[Polyisobutyl(18)]phenol as a PIB derivative (1 mmol) dissolved in DCM (5 mL) and the hydroxybenzotriazole (1.4 mmol) were added in succession. After 10 minutes the 1-ethyl-3-(3-dimethylaminopropyl)carbodiimide (EDC) (1.5 mmol) was added and the temperature of the reaction medium was allowed to rise slowly to ambient temperature (3 h or 16 h). The reaction medium was concentrated under reduced pressure. The cyclohexane was added to the residue, then washed three times with water or with a water/ethanol or water/acetonitrile mixture, and then with an aqueous solution saturated with sodium chloride. The organic phase was dried with Na₂SO₄ and filtered, and then the solvent was evaporated under reduced pressure.

### Example 2: Coupling Reaction Using EDC/DMAP

The N-protected amino acid (Fmoc-AA-OH or Boc-AA-OH) (1.3 mmol) was dissolved in DCM (5 mL) with magnetic stirring and in a nitrogen atmosphere, then cooled to 0° C. in an ice bath. The PIB derivative (free amine, alcohol, thiol, or phenol) (1 mmol) dissolved in DCM (5 mL) and 4-dimethylaminopyridine (DMAP) (0.3 mmol) were added in succession. After 10 minutes the EDC (2 mmol) was added and the temperature of the reaction medium was allowed to rise slowly to ambient temperature (3 h or 16 h). The reaction medium was concentrated under reduced pressure. The cyclohexane was added to the residue, then washed three times with water or with a water/ethanol or water/acetonitrile mixture, and then with an aqueous solution saturated with sodium chloride. The organic phase was dried with Na₂SO₄ and filtered, and then the solvent was evaporated under reduced pressure.

### Example 3: Deprotection of Fluorenylmethyl Carbamate (Fmoc)

The fluorenylmethyl carbamate derivative (1 mmol) was dissolved in DCM (5 mL) with magnetic stirring and was cooled to 0° C. in an ice bath. An ACN/HNEt2 solution (2:1) (5 mL) was added, then the reaction medium was stirred at ambient temperature for 4 h. The solvents were evaporated under reduced pressure. The cyclohexane was added to the residue, which was then washed three times with water or with a water/ethanol or water/acetonitrile mixture, and then with an aqueous solution saturated with sodium bicarbonate and with an aqueous solution saturated with sodium chloride. The organic phase was dried with Na₂SO₄ and filtered, and then the solvent was evaporated under reduced pressure.

### Example 4: Deprotection of Tert-Butoxycarbonyl (Boc)

The tert-butylcarbamate derivative (1 mmol) was dissolved in DCM (5 mL) with magnetic stirring and was cooled to 0° C. in an ice bath. A DCM/TFA mixture (1:1) (35 mL) was added, then the reaction medium was stirred at ambient temperature for 1 h. The solvents were evaporated under reduced pressure. The cyclohexane was added to the residue, which was then washed three times with water or with a water/ethanol or water/acetonitrile mixture, and then with an aqueous solution saturated with sodium bicarbonate and with an aqueous solution saturated with sodium chloride. The organic phase was dried with Na₂SO₄ and filtered, and then the solvent was evaporated under reduced pressure.

### Example 5: Detachment of the Peptide from the Anchoring Molecule

The tripeptide, anchored to its solid substrate (1 mmol) dissolved in DCM (5 mL) was added to a mixture of trifluoroacetic acid/triisopropylsilane/water (v/v/v, 95:2.5:2.5) (5 mL) previously cooled to 0° C. in an ice bath. The reaction medium was stirred for 3 h at ambient temperature. Ethyl ether was added to the reaction medium and the precipitate was collected by filtration and was dried in a vacuum.

What is claimed is:

1. A method for synthesizing peptides or proteins by successively elongating the second end (Nα) of a peptide chain of which the first end is attached to an anchoring molecule that is soluble in an apolar solvent by means of the carboxylic acid function (C-terminus) thereof or the amine function (Nα) thereof, characterized in that said anchoring molecule includes a polyolefin chain with at least 10 monomer units and preferably between 15 and 50 units.

2. The method according to claim 1, characterized in that said anchoring molecule only includes one polyolefin chain.

3. The method according to claim 1, characterized in that said polyolefin chain is a polyisobutene chain.

4. The method according to one of claim 1, characterized in that said anchoring molecule is a polyolefin.

5. The method according to one of claim 1, characterized in that said polyolefin chain is functionalized at one of the ends thereof.

6. The method according to one of claim 1, characterized in that said polyolefin chain comprises a number of unsaturated carbon-carbon bonds not exceeding 5% and preferably not exceeding 3%.

7. The method according to one of claim 1, characterized in that said polyolefin chain has an average molecular weight by mass of between 600 and 20,000, and preferably between 700 and 15,000.

8. The method according to one of claim 1, characterized in that said anchoring molecule includes a polyolefin chain (or is a polyolefin chain) terminated by a group selected from the group consisting of:
a —X function, where X is selected from the group consisting of: —OH, —NH₂, —SH;
a —Z—C₆H₄X¹ function, where
Z is O or is absent,
X¹ is selected from the group consisting of: —OH, —NH₂, —SH, —NH—NH₂, —CXRR¹, —C₆H₃R′(CRX)
(where X is selected from the group consisting of —OH, —NH₂, —SH, and R is selected from the group consisting of —H, Aryl, Heteroaryl, and R′ is selected from the group consisting of —H, -Alkyl, —O-Alkyl, -Aryl, —O-Aryl, Heteroaryl, —O-Heteroaryl),
a —CR″═CH—CHX function or a —CR″H—CH═CH—CHX function where X is selected from the group consisting of —OH, —NH₂, —SH, and R″ is methyl or ethyl.

9. The method according to one of claim 1, characterized in that:
said first end of said peptide chain is a first amino acid unit AA1, and
said peptide chain is formed of n amino acid units, and
the second end of said peptide chain is another amino acid unit AAn.

10. The method according to one of claim 1, characterized in that another amino acid unit AA(n+1) is added to said second end during said elongation.

11. The method according to one of claim 1 comprising at least one step wherein said peptide chain attached to said anchoring molecule is separated from the reaction medium by extraction in an apolar liquid.

12. The method according to claim 11, characterized in that the solubility of said peptide chain attached to said anchoring molecule in water is less than 30 mg/mL.

13. The method according to one of claim 3, characterized in that said anchoring molecule has a biobased carbon content greater than 90%, preferably greater than 93%, and even more preferably greater than 95%.
